(19) United States
(12) Patent Application Publication
(10) Pub. No.: US 2024/0350312 A1
(43) Pub. Date: Oct. 24, 2024

(54) Surgical Instrument And Method For Goniotomy Procedure

(71) Applicant: Nallakrishnan Family Trust, Willowbrook, IL (US)

(72) Inventor: Ravi Nallakrishnan, Willowbrook, IL (US)

(21) Appl. No.: 18/681,656

(22) PCT Filed: Aug. 1, 2022
(86) PCT No.: PCT/US2022/039003

§ 371 Date: Feb. 6, 2024

Related U.S. Application Data
(60) Provisional application No. 63/231,322, filed on Aug. 10, 2021.

(51) Int. Cl. A61F 9/007

(52) U.S. Cl. CPC A61F 9/00745

(57) ABSTRACT

An ophthalmic surgical instrument (10, 10B, 10C, 10D) includes a hand grip portion (12, 12B, 12C, 12D) having an elongated configuration with proximal and distal ends, and a tip portion (14, 14A, 14B, 14C, 14D) extending from the distal end. The tip portion (14, 14A, 14B, 14C, 14D) has the form of an elastomeric element (16, 16A, 16B, 16C, 16D) secured to the distal end of the hand grip portion (12, 12B, 12C, 12D) and a free end (18, 18A, 18B, 18C, 18D) for massaging the trabecular meshwork and outer wall of the canal of Schlemm in the eye to improve aqueous outflow for the reduction of intraocular pressure. In one form, the tip portion (14, 14A, 14B, 14C, 14D) has at least one irrigation passage (13) extending therethrough for directing an irrigation fluid proximate to the trabecular meshwork of the eye.

### PRIORITY

This application claims priority of U.S. Provisional Patent Application No. 63/231,322, filed Aug. 10, 2021, the entire contents of which is incorporated herein by reference.

### FIELD OF THE INVENTION

The present invention relates to a surgical instrument for performing ophthalmological procedures for treatment of eye diseases, such as glaucoma, and more particularly to a goniotomy surgical instrument having a tip portion with an elastomeric element having a roughened surface to massage the trabecular meshwork within the anterior chamber of the eye.

### BACKGROUND OF THE INVENTION

A goniotomy is a surgical procedure primarily used to treat congenital glaucoma. It is caused by a developmental arrest of some of the structures within the anterior (front) segment of the eye. These structures include the iris and the ciliary body, which produces the aqueous fluid needed to maintain the integrity of the eye. These structures do not develop normally in the eyes of patients with isolated congenital glaucoma. Instead, they overlap and block the trabecular meshwork, which is the primary drainage system for the aqueous fluid. As a result of this blockage, the trabecular meshwork itself becomes thicker and the drainage holes within the meshwork are narrowed. These changes lead to an excess of fluid in the eye, which can cause increased pressure that can damage the internal structures of the eye and cause glaucoma.

The purpose of a goniotomy is to clear the obstruction to aqueous outflow from the eye, which in turn lowers the intraocular pressure (IOP). Lowering the IOP helps to stabilize the enlargement of the cornea and the distension and stretching of the eye that often occur in congenital glaucoma. The size of the eye, however, will not return to normal. Most importantly, once the aqueous outflow improves, damage to the optic nerve is halted or reversed. The patient's visual acuity may improve after surgery.

The goniotomy procedure can restore normal drainage of aqueous humor from the eye by removing a full thickness segment of the trabecular meshwork, thus allowing the aqueous humor to drain through the open area from which the strip of trabecular meshwork has been removed. The goniotomy procedure and certain prior art instruments useable to perform such procedure are described In U.S. Pat. No. 6,979,328 and U.S. Patent Application Publication No. US 2018/0289544 A1, each of one of which is hereby incorporated by reference herein in its entirety.

At present there remains a need in the art for the development of simple, inexpensive, and accurate instruments useable to perform the procedure of massaging the tissues of the eye, including the trabecular meshwork and/or the canal of Schlemm to loosen or free deposits to assist in the restoration and/or improvement of the drainage of the eye.

### SUMMARY OF THE INVENTION

In accordance with one broad form of the present invention, a surgical instrument is disclosed which is particularly configured to facilitate performing a goniotomy such as for the treatment of glaucoma. The instrument includes a hand grip portion having an elongated configuration, with a proximal end and a distal end. The instrument further includes a tip portion which extends from, and which is operably connected with, the distal end of the hand grip portion. The tip portion has the form of an elastomeric element having a base that is secured to the distal end of the hand grip portion and a free end extending from the base. The tip portion includes at least one irrigation passage extending through the tip portion for directing an irrigation fluid proximate to the trabecular meshwork of the eye.

In another aspect of the present invention, the at least one irrigation passage terminates in a single aperture in the free end. Preferably, the single aperture is aligned with a central axis of the tip portion of the instrument. More preferably, the free end terminates in an annular surface surrounding the at least one irrigation passage.

In still another aspect of the present invention, the free end includes at least one relatively abrasive surface. In one preferred form of the invention.

In another aspect of the present invention, the abrasive of the free end is formed from an impregnated abrasive composition within or atop a surface of the elastomeric element. In one presently preferred form of the invention, the abrasive composition comprises diamond particles. In another preferred form of the invention, the relatively abrasive surface of the free end has the form of surface-roughening of the elastomeric element.

According to yet another aspect of the present invention, the elastomeric element is silicone.

In another broad form of the present invention, the instrument is assembled in combination with a vibratory handpiece connected to the hand grip portion, wherein the handpiece is configured to vibrate the free end in one of a torsional, longitudinal, and/or blended motion. In one preferred form, the handpiece is selected form on of the following handpieces: a longitudinally-vibrating phacoemulsification handpiece; a torsionally-vibrating phacoemulsification handpiece; an elliptically-vibrating phacoemulsification handpiece; a phacoemulsification handpiece configured for vibratory movement in three dimensions; a vitrectomy handpiece; a piezo electric handpiece; a solenoid valve handpiece; or a battery powered handpiece.

In another broad form of the present invention, the goniotomy surgical instrument is coupled with an irrigation supply source to provide an irrigation fluid (i) either through the instrument itself by way of one or more cannulas, or (ii) around the exterior of the instrument when coupled with an irrigation sleeve arranged around the exterior surface of the instrument. In one preferred form, the instrument is coupled with a vacuum source for aspirating fluids and/or tissues through one or more cannulas in the instrument or around the exterior of the instrument with an accompanying sheath or sleeve.

In one preferred form of the present invention, the hand grip portion of the instrument includes a reservoir for containing an irrigation fluid and a button to permit the user to selectively provide a burst or jet of irrigation fluid (i) through the instrument, or (ii) around the instrument when coupled with an irrigation sleeve arranged around a portion of the instrument.

In still another form of the present invention, the tip portion defines a central axis, and the free end terminates in a substantially flat configuration that is transverse to the central axis. The tip portion further includes a pair of oppositely facing, relatively abrasive, surfaces located on either side of the central axis.

In another form of the present invention, the tip portion overlies a length of the hand grip portion distal end.

In one preferred form of the invention, the free end terminates in a surface surrounding the at least one irrigation passage, and the surface includes a pair of opposite arcuate sides and a pair of opposite flat sides.

According to yet another form of the present invention, the tip portion defines a central axis and includes a pair of oppositely facing relatively abrasive surfaces located on either side of the central axis, wherein each one of the pair of oppositely facing relatively abrasive surfaces is concave. Preferably, each one of the pair of oppositely facing relatively abrasive surfaces is substantially elliptical. Preferably, each one of the pair of oppositely facing relatively abrasive surfaces extends along more than half of an axial length of the tip portion in the direction of the central axis.

According to yet another form of the present invention, the free end has a pair of relatively smooth, non-abrasive top and bottom surfaces and a pair of relatively abrasive, opposite lateral surfaces.

In still another broad form of the present invention, a method for improvement of the drainage of fluid of the eye includes the step of obtaining any of the surgical instruments disclosed herein and contacting the trabecular meshwork of the eye with the free end of the elastomeric element to massage the trabecular meshwork to dislodge deposits within the meshwork. The method further includes the step of introducing or directing an irrigating fluid into or through the surgical instrument to direct flow of the irrigating fluid from at least one irrigation passage in the elastomeric element or around the elastomeric element to the trabecular meshwork.

According to another broad form of the present invention, a surgical instrument is disclosed which is particularly configured to facilitate performing a goniotomy such as for the treatment of glaucoma. The instrument includes a hand grip portion having an elongated configuration, with a proximal end and a distal end. The instrument further includes a tip portion which extends from, and which is operably connected with, the distal end of the hand grip portion. The tip portion has the form of an elastomeric element having a base that is secured to the distal end of the hand grip portion and a free end extending from the base configured to mechanically stretch the trabecular meshwork of the eye.

In one preferred form, the tip portion defines a central axis, and the elastomeric element includes at least one roughened surface. Preferably, the roughened surface includes a pair of semi-elliptical roughened surfaces located on opposite sides of the central axis and a substantially rectangular roughened surface located between each one of the pair of semi-elliptical roughened surfaces.

### BRIEF DESCRIPTION OF THE DRAWINGS

In the accompanying drawings forming part of the specification, in which like numerals are employed to designate like parts throughout the same.

FIG. 1 is an isometric view, from above, of a first embodiment of a surgical instrument according to the present invention, and FIG. 1 shows the instrument having a reflux irrigating type handle or hand piece for permitting the user to introduce an amount of irrigating fluid through the instrument to the meshwork of the eye;

FIG. 2 is a left side elevation view of the instrument of FIG. 1;

FIG. 3 is a front elevation view of the instrument of FIG. 1;

FIG. 4 is a greatly enlarged, fragmentary, detailed isometric view of the distal portion of the instrument of FIG. 1, and FIG. 4 shows the operative, distal end of the instrument with an irrigation flow path terminating in the distal end of the instrument;

FIG. 5 is a greatly enlarged, fragmentary, detailed left side elevation view of the distal portion of the instrument circled in FIG. 2, and FIG. 5 shows the operative, distal end of the instrument with a pair of relatively abrasive, elliptical surfaces for massaging the trabecular meshwork of the eye;

FIG. 6 is a greatly enlarged, fragmentary, detailed top plan view of the distal portion of the instrument circled in FIG. 2, and FIG. 2 shows the operative, distal end of the instrument with a relatively non-abrasive, smooth surface extending between the two relatively abrasive concave surfaces for massaging the trabecular meshwork of the eye;

FIG. 7 is a greatly enlarged, fragmentary, isometric view taken from above of a second embodiment of a surgical instrument according to the present invention, and FIG. 7 shows only a distal tip portion of the instrument;

FIG. 8 is a front elevation view of the tip portion of the instrument shown in FIG. 7;

FIG. 9 is a left side elevation view of the tip portion of the instrument shown in FIG. 7;

FIG. 10 is a top plan view of the tip portion of the instrument shown in FIG. 7;

FIG. 11 is a greatly enlarged, fragmentary, isometric view taken from above of a third embodiment of a surgical instrument according to the present invention, and

FIG. 11 shows only the distal, operative portions of the instrument;

FIG. 11A is a greatly enlarged, fragmentary, isometric view taken from above of another version of the third embodiment of a surgical instrument according to the present invention, and FIG. 11A shows only the distal, operative irrigating portions of the instrument;

FIG. 12 is a greatly enlarged, isometric view taken from above of only the elastomeric, tip portion of the instrument of FIG. 11;

FIG. 13 is a greatly simplified, diagrammatic view of the instrument of the present invention in combination with a handpiece, and optionally in combination with an irrigation fluid supply, and further optionally in combination with an irrigation sleeve and a vacuum source;

FIG. 14 is an isometric view, from above, of a fourth embodiment of a surgical instrument according to the present invention, and FIG. 14 shows the instrument having a reflux irrigating type handle or hand piece for permitting the user to introduce an amount of irrigating fluid through the instrument to the meshwork of the eye;

FIG. 15 is a greatly enlarged, isometric view taken from above of only the elastomeric, tip portion and irrigation sleeve of the instrument of FIG. 14;

FIG. 16 is a greatly enlarged, cross-sectional view taken in a transverse plane relative to the central axis of the tip portion, and FIG. 16 shows the solid elastomeric element fitted within a smaller chamber of the hollow irrigation sleeve;

FIG. 17 is a right-side elevational view of a fifth embodiment of a surgical instrument according to the present invention, and FIG. 17 shows a non-irrigating embodiment of the instrument;

FIG. 18 is a partial, cross-sectional view taken in a vertical plane containing the central axis of a portion of the instrument shown in FIG. 17;

FIG. 19 is a greatly enlarged, fragmentary, cross-sectional view taken in a vertical plane containing the central axis of a portion of the instrument shown in FIG. 17;

FIG. 19A is a greatly enlarged, fragmentary, cross-sectional view taken in a vertical plane containing the central axis of a portion of an irrigating variation of the instrument shown in FIG. 17;

FIG. 20 is a greatly enlarged, fragmentary, isometric view, taken from above and the left side, of the operative, tip portion of the instrument of FIG. 17;

FIG. 21 is a greatly enlarged, fragmentary, front elevational view, of the operative, tip portion of the instrument of FIG. 17;

FIG. 22 is a greatly enlarged, fragmentary, right side elevational view of a portion of the instrument of FIG. 17;

FIG. 23 is a greatly enlarged, fragmentary, top plan view of a distal portion of the instrument of FIG. 17; and

FIG. 23A is a greatly enlarged, fragmentary, top plan view of a distal portion of the irrigating version of the instrument of FIG. 19A.

### DESCRIPTION OF THE PREFERRED EMBODIMENTS

While this invention is susceptible of embodiment in many different forms, this specification and the accompanying drawings disclose only specific forms as examples of the invention. The invention is not intended to be limited to only the embodiments so described, and the scope of the invention will be pointed out in the appended claims.

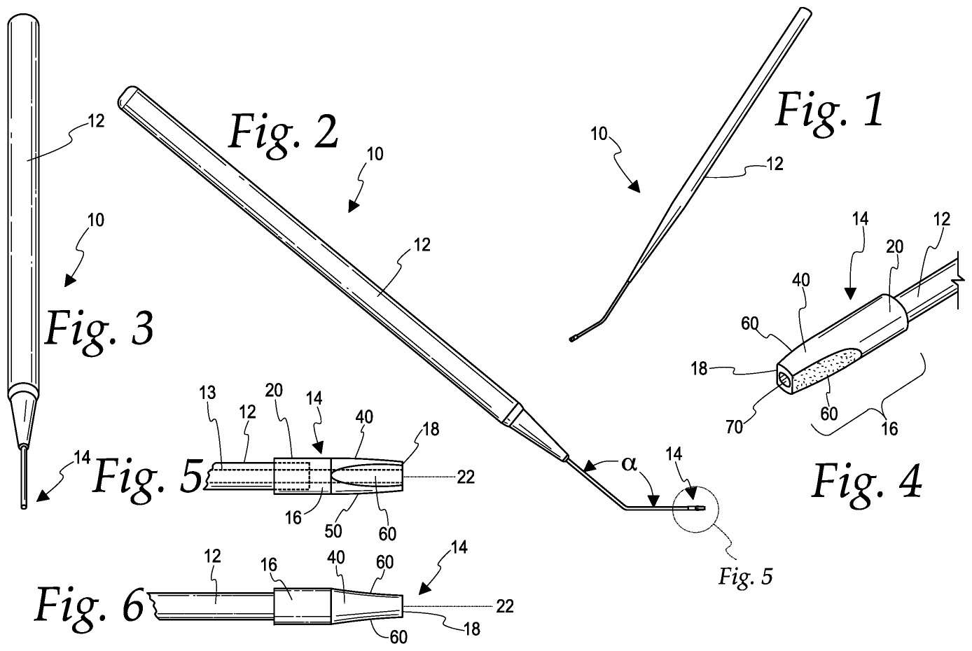

A first embodiment of a goniotomy surgical instrument of the present invention is illustrated in FIGS. 1-6 and 13, wherein the instrument is designated generally by the reference number 10. The first illustrated embodiment of the instrument 10 includes an elongated handpiece or hand grip portion 12 for being gripped by a user of the instrument 10, and which can be provided with either a rounded configuration as illustrated or with a flattened, polygonal, or irregular configuration (not illustrated). The hand grip portion 12 has a proximal end and a distal end.

The hand grip portion 12 includes an internal reservoir of an irrigation fluid or a connection to an external irrigation fluid supply source and a through passage 13 that communicates to the instrument operative, distal end to permit flow of an irrigating fluid through the instrument 10 to a target location proximate to the trabecular meshwork. The hand grip portion 12, or other machinery or system connected to the hand grip portion 12, may include a pressure switch, collapsible wall, button, bellows, etc. or conventional or non-conventional means for actuating the instrument 10 to cause an on-demand or selective flow or reflux of irrigating fluid to be expelled from one or more ports or outlets in the tip portion of the instrument to a target location. There are many commercially available irrigating handpieces or systems on the market, and it will be understood that the instrument 10 may be adapted to function with such handpieces or systems.

Referring to FIG. 2, the hand grip portion 12 proximal and distal sections or portions are arranged at an obtuse angle (a) relative to each other of between about 120 degrees and about 140 degrees, and more preferably at an angle (a) of about 130 degrees.

The instrument 10 includes a specifically configured massaging or gentle abrading tip portion 14, extending from the distal end of the hand grip portion 12, which facilitates scraping, rubbing, massaging, and/or abrading the tissues of the eye to permit drainage of aqueous humor to enhance the vision of the patient and to reduce pressure in the eye.

With reference to FIGS. 4-6, the tip portion 14 of the instrument 10 comprises a hollow elastomeric element 16 having a cylindrical base portion or base 20 secured around the distal end of the hand grip portion 12, and a distalmost portion or free end 18 extending from the base 20. In accordance with the invention, the elastomeric element 16 has one or more relatively abrasive surfaces, which will be discussed in greater detail hereinafter.

With reference to FIGS. 5 and 6, the instrument elastomeric element 16 includes a pair of substantially elliptical (i.e., mostly forming the shape of an ellipse, but having a truncated, flat end) relatively abrasive surfaces 60 that are thought to more safely and effectively free or dislodge deposits from the tissues around the Schlemm's canal compared to prior art devices. The elastomeric element 16 further includes an upper sloping portion 40 and lower sloping portion 50 that are smooth and substantially free of any abrasive or roughened surface. The relatively abrasive surfaces 60 are preferably located on the lateral sides to the elastomeric element 16, in between the upper sloping portion 40 and the lower sloping portion 50. The relatively abrasive surfaces 60 may further include a plurality of protrusions extending outwardly therefrom (not illustrated) for improved abrasion of the eye tissues.

The inventors have found that the instrument 10 may be more effective in restoring the normal drainage, or at least sufficient drainage, through the trabecular meshwork with the abrasion of the clogged or blocked areas of the eye, while presenting only the non-abrasive surfaces against the endothelium. In some applications, the instrument 10 may be operated by hand or may be part of a larger device, such as a vibratory, piezoelectric handpiece or other movable handpiece.

With reference to FIGS. 5 and 6, it can be seen that the elastomeric element 16 tapers radially inwardly (e.g., narrows) in a direction away from the base 20 moving distally along a central axis 22 extending through the geometric center of the element 16.

In one preferred form of the first embodiment of the instrument 10, the elastomeric element 16 has a total length extending from the handle 12 in the direction along the central axis 22 of about 2.5 mm, with the base 20 having an axial length of about 1.1 mm and tapering free end 18 containing the roughened surface having a length of about 1.4 mm (+/−0.2 mm). Preferably, the width or outer diameter of the elastomeric element 16 is about 0.7 mm at the base portion 20. The distal end of the hand grip portion 12 extends about 0.6 mm into the base portion 20 of the elastomeric element 16.

With reference now to FIG. 4, the free end 18 of the elastomeric element 16 includes at least one irrigation passage extending through the elastomeric element 16 for directing an irrigation fluid from the hand grip portion 12 proximate to the trabecular meshwork of the eye. Preferably, the irrigation passage has a circular cross-section in a transverse or normal plane relative to the central axis 22 (FIG. 5) and is centered on the central axis 22. The irrigation passage terminates in one or more ports or apertures 70 in the substantially flat free end 18.

In order to effect the desired massaging/abrading action of the instrument 10, the relatively abrasive surfaces 60 of the elastomeric element 16 include an impregnated abrasive composition. The impregnated abrasive composition may comprise diamond particles or abrasive granular or dust particles. In one presently preferred form of the invention, the diamond particles have an approximate size of about 30 microns. Alternatively, the abrasive surface may be provided through subjecting the elastomeric element 16 to a secondary coating process. In yet another alternative, the relatively abrasive surface of the elastomeric element 16 is formed from a surface-roughening or texturing of the elastomeric material of the element 16.

In one presently preferred form of the invention, the relatively abrasive surfaces 60 of the instrument 10 have a surface roughness of between about N8-N12 according to the ISO 1302 Roughness Grade Numbers. Preferably, the elastomeric element 16 is formed from a medical grade silicone material with a durometer of between about 60 and about 90 on the Shore 00 scale, while the hand grip portion 12 of the instrument is formed from a metal or sufficiently stiff plastic or composite material. One preferred material of the elastomeric element 16 is silicone USP Class VI, 60A.

The handle portion 12 of the instrument 10 is preferably made of material like malleable nitinol or other malleable alloys or metals and is provided with a tip portion 14 in the form of an elastomeric element 16 having a base 20, secured overtop of the distal end of the hand grip portion 12, and a free end 18 extending from the base 20. Notably, the free end 18 is provided with a relatively abrasive surface, compared to the material of the base portion 20 of the elastomeric element 16. The elastomeric element 16 may be secured to the handle portion 12 of the instrument 10 by mechanical fit, overmolding or bi-injection molding, adhesive, welding, crimping, or any other suitable means. The elastomeric element 16 may further be removable from the handle portion 12 for replacement by way of mating threads, locks, etc.

The instrument 10 may be a single use instrument that is intended to be discarded after it is used in a surgical procedure. However, if the instrument 10 is to be a multi-use instrument, which would be sterilized between uses, then the elastomeric element 16 may be removable from the irrigating handle or hand grip portion 12. The instrument 10 may also be made for single-use, disposable type, and/or may be formed from appropriate materials that can be sterilized and subject to Ethylene oxide or Gamma radiation or other sterilizations and that degrades when subjected to steam sterilization.

In one method of operation of the instrument 10, the user would create two incisions or ports within the eye. The ports are used to accommodate the insertion of the instrument 10, and the implantation and removal of viscoelastic substances (OVD) from the eye, such as with an irrigation/aspiration instrument or instruments. The ports would be used to fill the anterior chamber of the eye with viscoelastic material to the appropriate viscosity. The instrument 10 into the first port to gently rub the area of the trabecular meshwork, about 120 degrees, with the free end 18 of the tip 14 about three to five times to free or dislodge deposits from the trabecular meshwork. A MORI Goniotomy lens may be used to observe the area being rubbed. The ports would be used to remove the viscoelastic material from the anterior chamber of the eye. The anterior chamber may be filled with balanced salt solution (BSS) to reach the desired intraocular pressure. In one preferred form of the method, the side ports in the eye are formed at the 12 o'clock and 3 o'clock positions. In another preferred form of the method of use of the instrument 10, the side ports in the eye are formed at the 10 o'clock and 3 o'clock positions, and a third port is formed at the 12 o'clock position to accommodate insertion of an intraocular lens (IOL). The user of the instrument 10 may selectively release a pulse or reflux of irrigation fluid through the passage 13 of the hand grip portion 12 and through the irrigation passage distal aperture 70 by engaging a means (e.g., flexible switch or button) to increase pressure within the reservoir of the hand grip portion 12 to send a jet or jets of the fluid to massage the Schlemm's canal or trabecular meshwork tissues.

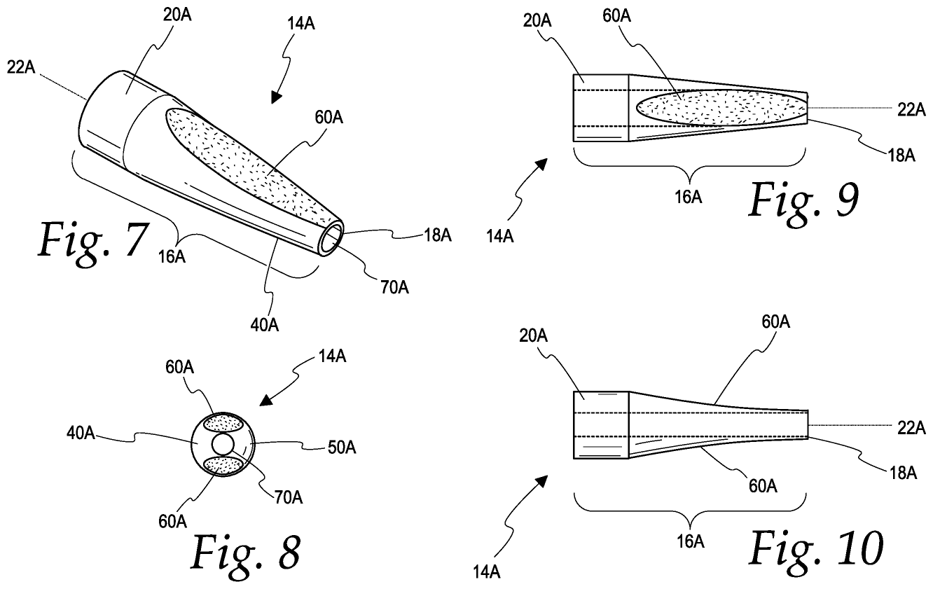

A second embodiment of the operative distal end of a goniotomy surgical instrument of the present invention is illustrated in FIGS. 7-10, wherein only the distal end or portion 14A of the instrument is illustrated. It will be understood that the remainder of the instrument and the handle would be the same as discussed above with respect to the hand grip portion 12 and the instrument 10. The numbered features of the second embodiment of the instrument illustrated in FIGS. 7-10 are designated generally with the suffix letter “A” and are analogous to features of the first embodiment of the instrument 10 that share the same number (without the suffix letter “A”).

The second illustrated embodiment of the instrument is similar in nature to the first illustrated embodiment of the instrument 10 and includes a hand grip having a proximal and distal end with a tip portion 14A extending from the distal end of the hand grip portion. The tip portion 14A comprises a hollow, tubular elastomeric element 16A having a base 20A secured around (or inside of) the distal end of the hand grip portion, and a tapering free end 18A extending from the base 20A.

With reference to FIGS. 9 and 10, the tip portion 14A differs from that of the previously discussed embodiment of the instrument 10, in that the tip portion 14A includes an elastomeric element 16A with a different tapering configuration such that the abrasive, concave surfaces 60A account for about 75% of the total length of the elastomeric element 16A, as measured along the central axis 22A. In one preferred form, the elastomeric element 16A has a total length of about 2.5 mm, with the base 20A having an axial length of about 0.23 mm and tapering free end 18A containing the roughened surfaces 60A having a length of about 1.91 mm (+/−0.2 mm). Preferably, the width or outer diameter of the elastomeric element 16A is about 0.7 mm at the base portion 20A.

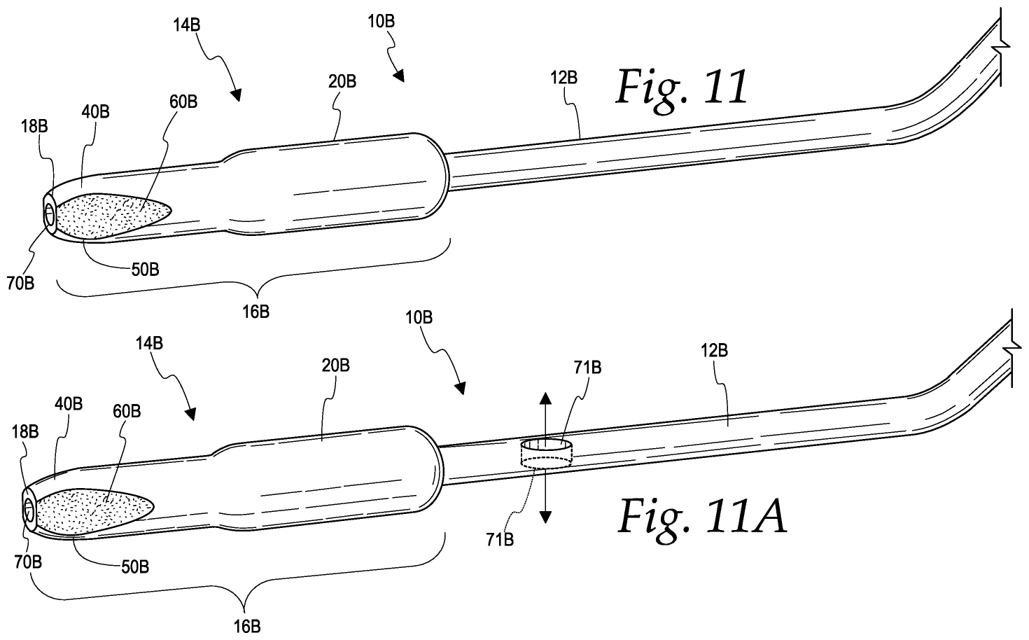

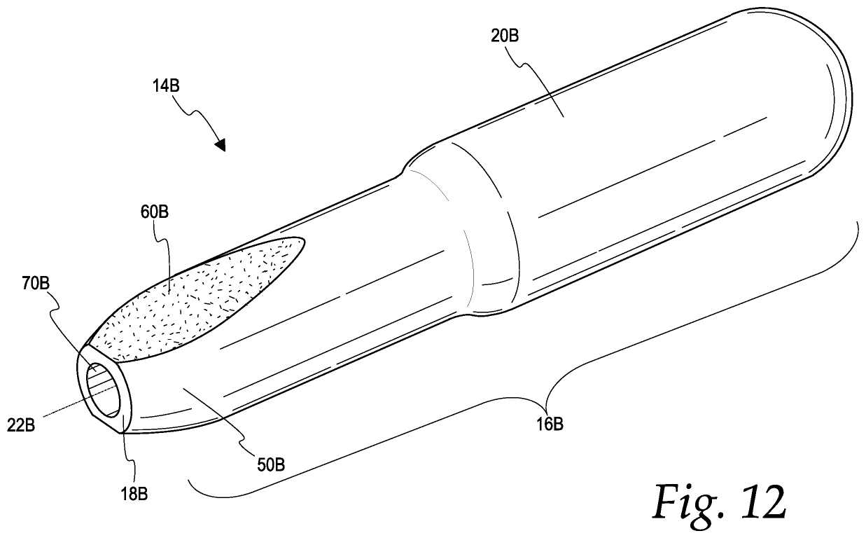

A third embodiment of a goniotomy surgical instrument of the present invention is illustrated in FIGS. 11 and 12, wherein only the distal end or portion 14B of the instrument is illustrated. The numbered features of the third embodiment of the instrument illustrated in FIGS. 11 and 12 are designated generally with the suffix letter “B” and are analogous to features of the first embodiment of the instrument 10 that share the same number (without the suffix letter “B”).

The third illustrated embodiment of the instrument is similar in nature to the first illustrated embodiment of the instrument 10 and includes a hand grip (e.g., 12) having a proximal and distal end with a tip portion 14B extending from the distal end of the hand grip portion. The tip portion 14B comprises a hollow, tubular elastomeric element 16B having a base 20B secured around the distal end of the hand grip portion, and a tapering free end 18B extending from the base 20B.

With reference to FIGS. 11 and 12, the tip portion 14B differs from that of the previously-discussed embodiment of the instrument 10, in that the tip portion 14B includes an elastomeric element 16B with a different configuration whereby the base 20B and the free end 18B are substantially cylindrical bodies, and the there is a step between the base 20B and the free end 18B. Furthermore, the free end 18B only tapers at location proximate to the irrigation passage distal port or aperture 70B located at the distalmost end of the instrument.

The particular arrangement of the tip portion 14B is believed to produce an advantageous for manufacturing of a more robust tip having irrigating capabilities.

With reference to FIG. 11A, an alternate version or configuration of the instrument 10B is illustrated, which has a pair of opposite, lateral irrigation ports or apertures 71B located on either side of the instrument handle 12B distal end for providing a lateral flow of irrigation fluid to, or around, the target surgical site or area.

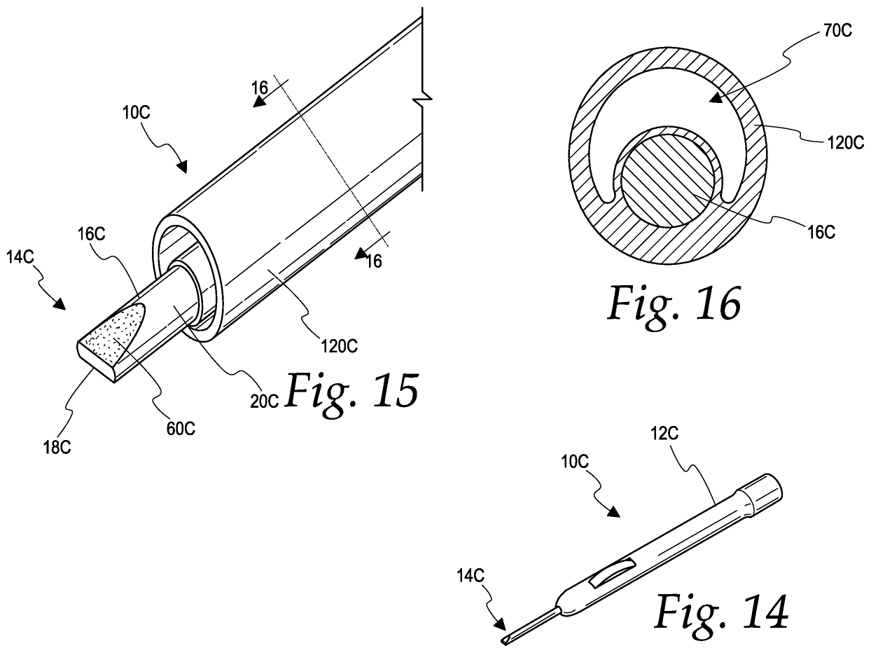

A fourth embodiment of a goniotomy surgical instrument of the present invention is illustrated in FIGS. 14-16, wherein the instrument is designated by the reference numeral 10C. The numbered features of the fourth embodiment of the instrument illustrated in FIGS. 14-16 are designated generally with the suffix letter “C” and are analogous to features of the first embodiment of the instrument 10 that share the same number (without the suffix letter “C”).

The fourth illustrated embodiment of the instrument 10C is similar in nature to the first illustrated embodiment of the instrument 10 and includes a hand grip 12C having a proximal and distal end with a tip portion 14C extending from the distal end of the hand grip portion 12C. However, the tip portion 14C comprises a solid, tubular elastomeric element 16C having a base 20C secured around the distal end of the hand grip portion 12C, and a tapering free end 18C extending from the base 20C. The elastomeric element 16C includes one or more roughened surfaces 60C for massaging or abrading the tissues of the eye to improve drainage through the Schlemm's Canal.

With reference to FIGS. 15 and 16, the tip portion 14C differs from that of the previously-discussed embodiment of the instrument 10, in that the tip portion 14C utilizes an exterior irrigation sleeve 120C in order to provide an irrigation passage terminating in an aperture or port 70C in communication with the fluid supply reservoir in the hand grip portion 12C or external fluid source. As can be seen in FIG. 16, the solid elastomeric element 16C is fitted within one chamber of the sleeve 120C while a larger, second chamber defines part of the irrigation passage which terminates in the crescent-shaped aperture 70C that partially surrounds the free end 18C.

The particular arrangement of the tip portion 14C of the instrument 10C is believed to produce improved irrigation by increasing the flow output of the reflux type irrigating hand grip portion 12C and may be more easily manufactured compared to the embodiments discussed above.

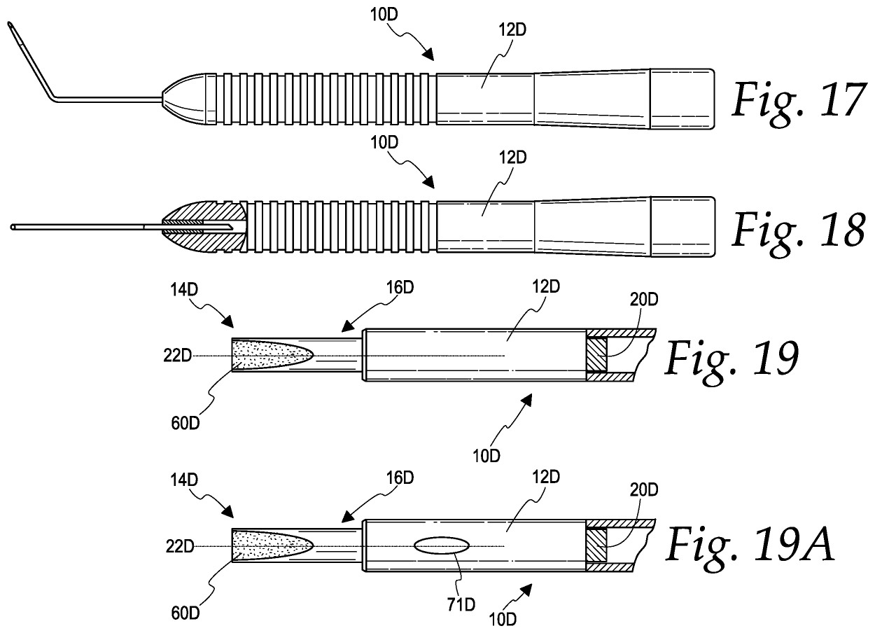

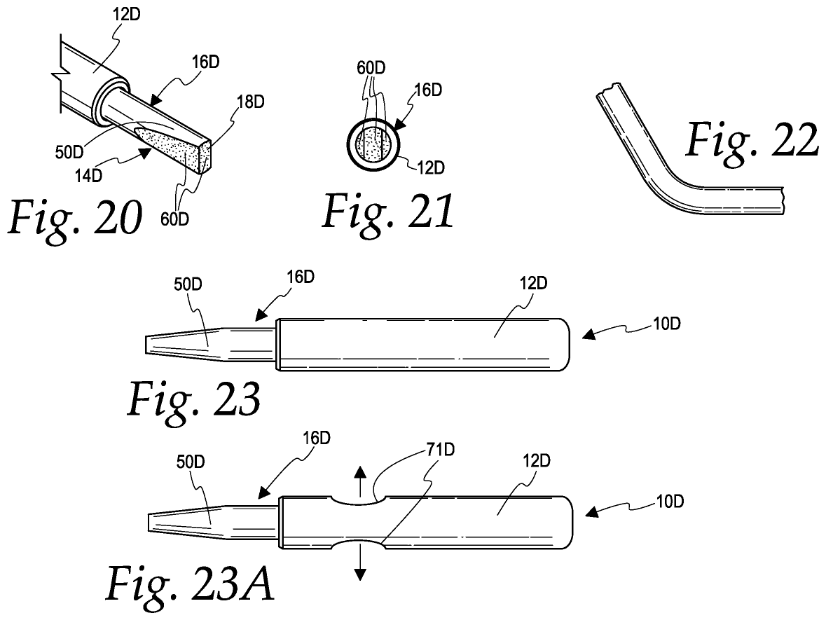

A fifth embodiment of a goniotomy surgical instrument of the present invention is illustrated in FIGS. 17-23, wherein the instrument is designated by the reference numeral 10D. The numbered features of the fifth embodiment of the instrument illustrated in FIGS. 17-23 are designated generally with the suffix letter “D” and are analogous to features of the first embodiment of the instrument 10 that share the same number (without the suffix letter “D”).

The fifth illustrated embodiment of the instrument 10D is similar in nature to the first illustrated embodiment of the instrument 10 and includes a hand grip 12D having a proximal and distal end with a tip portion 14D extending from the distal end of the hand grip portion 12D. However, the tip portion 14D comprises a solid, tubular elastomeric element 16D having a base 20D secured at the distal end of the hand grip portion 12D, and a tapering free end 18D extending from the base 20D along a central axis 22D. The elastomeric element 16D includes a pair of opposite smooth surfaces 50D, and three contiguous roughened surfaces 60D (two semi-elliptical lateral surfaces, and one rectangular end surface therebetween) for massaging, stretching, and/or abrading the tissues of the eye to improve drainage through the Schlemm's Canal.

The particular arrangement of the tip portion 14D of the instrument 10D has been found by the inventors to cause cellular stimulation of the Schlemm's Canal, stretching the meshwork mechanically in a low cost, effective manner that is comparable to stimulation caused by selective laser trabeculoplasty (“SLT”), which can result in improved drainage and lowered intraocular pressure. It is believed that stimulation by mechanical stretching promotes pro-inflammatory cytokines to be produced by endothelial cells to increase egress of aqueous humor from the eye.

With reference to FIGS. 19A and 23A, an irrigating version of the instrument 10D is illustrated, which has a pair of opposite, lateral irrigation ports 71D located on either side of the instrument handle 12D distal end for providing a lateral flow of irrigation fluid to the target area. It will be understood that the instrument 10D may have only one aperture or port 71D, or more than two ports 71D for some applications.

The inventors have further determined that it may further be advantageous to combine any one of the above-discussed instruments disclosed herein with an irrigation system and/or aspiration system. For example, the instruments may be incorporated into an aspiration/irrigation handpiece or system to supply irrigating liquids to the eye in order to aid in flushing and aspirating dislodged particles in the eye during the use of the instruments.

It will further be understood that the instruments disclosed herein may be incorporated into a larger machine or device, whereby the hand grip portion is connected to such a machine or device, and may be controlled, operated, or manipulated by such a machine or device and not necessarily by hand.

In some applications, the above-discussed elastomeric elements may have a different non-cylindrical form, such as polygonal shapes or irregular shapes which may be more robust during use.

In still other applications, the instruments disclosed herein (or at least a portion thereof) may be formed from a material that is malleable, such as a metal, wire, or polymer, which may be bent by a user to hold its position for better manipulation of the instrument by the user. For example, the hand grip portion may be formed from a malleable metal that may be selectively bent by a user during different uses of the instrument to reach different locations of the target areas of the eye.

The operative, distal end of the instruments disclosed herein are designed for engagement with the tissues of the eye proximal to the trabecular meshwork and/or the Canal of Schlemm. The relatively roughened of the surface or surfaces of the elastomeric element favors a massaging effect on the tissues to free or dislodge possible ‘clogs’ or deposits and stretch the tissue to stimulate cells in the meshwork to facilitate better flow that helps in reducing the intra-ocular pressure (IOP). Increased IOP can lead to permanent blindness, caused in cases of patients suffering with glaucoma or other diseases that cause the blockage of the Canal. It will be understood that the other instruments discussed herein would function similarly.

In other forms of the invention, at least the tip portion of the instruments discussed above may be removable from the remaining portion of the instrument for disposal, replacement, cleaning, and/or other modification (retractable for improved entry into the incision, safety). In still other forms of the present invention, the tip portion may be wholly retractable within the handle portion or hand grip portion of the instrument, and may be extended beyond the handle portion by the user after the instrument has been inserted to the target area in the eye to offer improved safety.

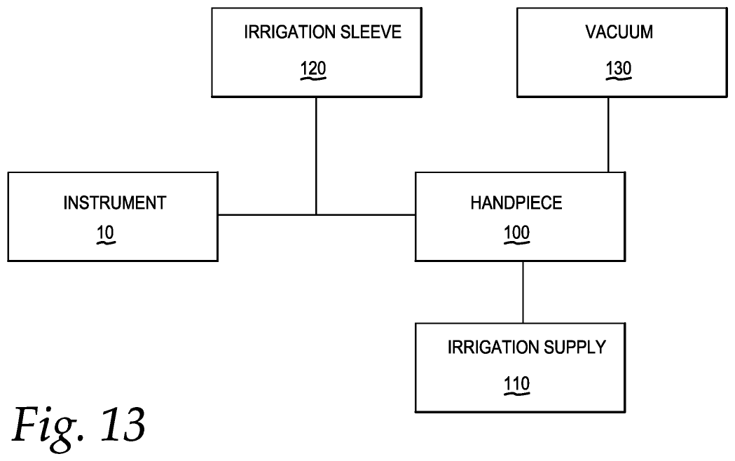

With reference to FIG. 13, in another broad form of the invention, each of the instruments disclosed herein is capable of being attached or otherwise coupled with a movable or vibratory handpiece 100 to assist in performing the goniotomy procedure. The handpiece 100 may be, for example, a longitudinally-vibrating phacoemulsification handpiece, a torsionally-vibrating phacoemulsification handpiece, an elliptically-vibrating phacoemulsification handpiece, a phacoemulsification handpiece configured for vibratory movement in three dimensions, a vitrectomy handpiece, a piezo electric handpiece, an ultrasound handpiece, a solenoid valve handpiece, pneumatic, or a battery powered handpiece. Other vibratory handpieces may be used with the instruments disclosed herein.

The handpiece 100 may be developed for the treatment of Open Angle Glaucoma, specifically at the Juxtacanalicular Space (JCS), but not limited to, or based on, combination treatments including vibrating, pulsating, oscillating, Guillotine, Piezo, Radiofrequency (RF), Neodymium-doped yttrium aluminum garnet (Nd:YAG) laser platforms with specially designed tips and/or laser probes.

The proximal end or portion of the instrument would not function as a hand grip, per se, when incorporated into a handpiece 100 that is gripped by a user, and the proximal portion of the instrument may be removably or non-removably coupled with the handpiece 100, such as by mating threads, luer lock, force fit, snap-fit, etc.

In some applications, the goniotomy surgical instruments disclosed herein may be coupled with an irrigation fluid supply source 110 to provide an irrigation fluid either (i) through one or more cannulas in the instruments, or (ii) around the exterior surface of the tip portion of the instruments when coupled with an irrigation sleeve 120 arranged around a portion of the distal end of the instrument. In some applications, the goniotomy surgical instruments disclosed herein may be coupled with a vacuum generator (shown diagrammatically as 130 in FIG. 11) to provide aspiration of fluids or tissues either (i) through one or more cannulas in the instruments, or (ii) around the exterior surface of the tip portion of the instruments.

Other features and advantages will be readily apparent from the following the accompanying drawings and the appended claims.

1. A surgical instrument for massaging the trabecular meshwork of the eye, the instrument comprising:
a hand grip portion having an elongated configuration, having proximal and distal ends; and
a tip portion extending from the distal end of said hand grip portion,
said tip portion comprising an elastomeric element having a base secured to said distal end of said hand grip portion, and a tapered free end extending from said base,
said free end configured to massage the trabecular meshwork of the eye, and at least one irrigation passage extending through said tip portion for directing an irrigation fluid proximate to the trabecular meshwork of the eye.

2. The surgical instrument in accordance with claim 1 wherein said at least one irrigation passage terminates in a single aperture in said free end.

3. The surgical instrument in accordance with claim 2 wherein said single aperture is aligned with a central axis of said tip portion.

4. The surgical instrument in accordance with claim 1 wherein said free end includes at least one relatively abrasive surface.

5. The surgical instrument in accordance with claim 4 wherein said at least one relatively abrasive surface of said free end comprises an impregnated abrasive composition.

6. The surgical instrument in accordance with claim 4 wherein said relatively abrasive surface of said free end comprises surface-roughening.

7. The surgical instrument in accordance with claim 1 in combination with a handpiece connected to said hand grip portion configured to vibrate said free end.

8. The surgical instrument in accordance with claim 7 wherein said handpiece is one of: a longitudinally-vibrating phacoemulsification handpiece; a torsionally-vibrating phacoemulsification handpiece, an elliptically-vibrating phacoemulsification handpiece, a phacoemulsification handpiece configured for vibratory movement in three dimensions, a vitrectomy handpiece, a piezo electric handpiece, a solenoid valve handpiece, or a battery powered handpiece.

9. The surgical instrument in accordance with claim 1 in combination with an irrigation supply source to provide an irrigation fluid (i) through said at least one irrigation passage of said instrument.

10. The surgical instrument in accordance with claim 1 in combination with a vacuum to provide aspiration through said instrument, or around said instrument.

11. The surgical instrument in accordance with claim 1 wherein said hand grip portion includes a reservoir for containing an irrigation fluid and a means to selectively provide an irrigation fluid through said at least one irrigation passage of said instrument.

12. The surgical instrument in accordance with claim 1 wherein said tip portion defines a central axis and said free end is substantially flat and transverse to said central axis, said tip portion further includes a pair of oppositely facing relatively abrasive surfaces located on either side of said central axis.

13. The surgical instrument in accordance with claim 1 wherein said tip portion overlies a length of said hand grip portion distal end.

14. The surgical instrument in accordance with claim 1 wherein said free end terminates in an annular surface surrounding said at least one irrigation passage.

15. The surgical instrument in accordance with claim 1 wherein said free end terminates in a surface surrounding said at least one irrigation passage, said surface having a pair of opposite arcuate sides and a pair of opposite flat sides.

16. The surgical instrument in accordance with claim 1 wherein said tip portion defines a central axis and includes a pair of oppositely facing relatively abrasive surfaces located on either side of said central axis, each one of said pair of oppositely facing relatively abrasive surfaces is concave.

17. The surgical instrument in accordance with claim 16 wherein each one of said pair of oppositely facing relatively abrasive surfaces is substantially elliptical.

18. The surgical instrument in accordance with claim 16 wherein each one of said pair of oppositely facing relatively abrasive surfaces extends along more than half of a length of said tip portion in the direction of said central axis.

19. The surgical instrument in accordance with claim 1 wherein said free end has a pair of relatively smooth, non-abrasive top and bottom surfaces and a pair of relatively abrasive, opposite lateral surfaces.

20. A method for improvement of the drainage of fluid of the eye, wherein the method comprises the steps of:
obtaining the surgical instrument in accordance with claim 1;
contacting the trabecular meshwork of the eye with said tapered free end of said elastomeric element to massage the trabecular meshwork to dislodge deposits within the meshwork; and
directing an irrigating fluid through said surgical instrument to direct flow of the irrigating fluid from said at least one irrigation passage to the trabecular meshwork.

21. A surgical instrument for massaging the trabecular meshwork of the eye, the instrument comprising:
a hand grip portion having an elongated configuration, having proximal and distal ends; and
a tip portion extending from the distal end of said hand grip portion,
said tip portion comprising an elastomeric element having a base secured to said distal end of said hand grip portion, and a tapered free end extending from said base, said free end configured to mechanically stretch the trabecular meshwork of the eye.

22. The surgical instrument in accordance with claim 21 wherein said tip portion defines a central axis and said elastomeric element includes at least one roughened surface.

23. The surgical instrument in accordance with claim 22 wherein said at least one roughened surface includes a pair of semi-elliptical roughened surfaces located on opposite sides of said central axis and a substantially rectangular roughened surface located between said pair of semi-elliptical roughened surfaces.

24. The surgical instrument in accordance with claim 21 in combination with a handpiece connected to said hand grip portion configured to vibrate said free end.

25. The surgical instrument in accordance with claim 24 wherein said handpiece is one of: a longitudinally-vibrating phacoemulsification handpiece; a torsionally-vibrating phacoemulsification handpiece, an elliptically-vibrating phacoemulsification handpiece, a phacoemulsification handpiece configured for vibratory movement in three dimensions, a vitrectomy handpiece, a piezo electric handpiece, a solenoid valve handpiece, or a battery powered handpiece.

26.-50. (canceled)
